(19) United States
(12) Patent Application Publication
(10) Pub. No.: US 2025/0025655 A1
(43) Pub. Date: Jan. 23, 2025

(54) NASAL INTERFACE APPARATUS WITH AIR ENTRAINMENT PORT OF ADJUSTABLE OPEN AREA

(71) Applicant: The Governors of the University of Alberta, Edmonton (CA)

(72) Inventors: Andrew MARTIN, Edmonton (CA); Cole CHRISTIANSON, Edmonton (CA); Ira KATZ, Edmonton (CA)

(21) Appl. No.: 18/905,533

(22) Filed: Oct. 3, 2024

Related U.S. Application Data
(62) Division of application No. 17/423,416, filed on Jul. 15, 2021.
(60) Provisional application No. 62/794,268, filed on Jan. 18, 2019.

(51) Int. Cl. A61M 16/06; A61M 16/10; A61M 16/20

(52) U.S. Cl. CPC A61M 16/0677; A61M 16/201; A61M 2016/1025; A61M 2202/0208

(57) ABSTRACT

A nasal interface apparatus is provided for delivering a gas to a human via a gas supply tube and a pair of tubular nasal inserts. The apparatus includes a manifold hollow body defining an internal chamber, an inlet for fluid communication from the gas supply tube into the internal chamber, an outlet for fluid communication between the internal chamber and the pair of nasal inserts, and an air entrainment port for fluid communication between the internal chamber and a space external to the hollow body. The apparatus also includes a valve member movable relative to the hollow body for varying the size of the open area of the air entrainment port. The open area of the air entrainment port may be varied to regulate a pressure signal detected by a pulse-flow oxygen concentrator (POC).

### CROSS-REFERENCE TO RELATED APPLICATION

This application claims priority to U.S. provisional patent application No. 62/794,268, filed on Jan. 18, 2019, the entire contents of which is incorporated herein by reference.

### FIELD OF THE INVENTION

The present invention relates to a nasal interface apparatus for delivering a gas to a patient via the patient's nostrils.

### BACKGROUND OF THE INVENTION

A nasal cannula is a device used to deliver supplemental oxygen to a patient via the patient's nostrils. A conventional nasal cannula includes a supply tube extending from a first end for connection to an oxygen source, to a second end that bifurcates to form a loop including a pair of tubular nasal prongs. To conserve oxygen, the oxygen source may be a portable pulse-flow oxygen concentrator (POC)—i.e., a portable machine configured to release an oxygen bolus into the supply tube only when the patient inhales, as detected by monitoring a pressure signal in the supply tube at an outlet of the POC. The nasal prongs fit loosely within the nostrils so as to define intra-nostril spaces between the nasal prongs and the nostril inner walls. When the patient inhales, the POC detects the resulting pressure signal, and releases an oxygen bolus into the supply tube. The patient inhales the oxygen bolus through the nasal prongs, along with room air entrained through the intra-nostril spaces. When the patient exhales, the patient exhales through the intra-nostril spaces.

The POC is typically used when the patent is awake and active, but not when the patient is sleeping. When sleeping, the patient's lower respiratory flow rate may be inadequate to generate the pressure signals needed to trigger pulse delivery from the POC. The POC could be configured to respond to lower pressure signals, but this increases the risk of false detection of patient inhalation, and suboptimal oxygen delivery. The POC could be configured with a “normal model” and a “sleep mode” with different pulse sensing and delivery settings, but this increases the complexity of the POC and its use. Accordingly, a patient that uses a pulse-flow POC during the day time, typically uses a continuous-flow stationary oxygen concentrator during the night time. From a cost and convenience perspective, it would be desirable if such a patient could use the pulse-flow POC during the night time as well.

### SUMMARY OF THE INVENTION

The present invention relates to a nasal interface apparatus for delivering a gas to a patient via the patient's nostrils. More particularly, the nasal interface apparatus of the present invention has an air entrainment port of adjustable open area, which allows for regulation of a pressure signal detected by a pulse-flow gas source, such as POC.

In one aspect, the present invention comprises a nasal interface apparatus for delivering a gas to a patient via a gas supply tube and a pair of tubular nasal inserts. The nasal interface apparatus comprises a manifold and at least one valve member. The manifold comprise a hollow body. The hollow body defines an internal chamber, at least one inlet for fluid communication from the gas supply tube into the internal chamber, at least one outlet for fluid communication between the internal chamber and the pair of nasal inserts, and at least one air entrainment port for fluid communication between the internal chamber and a space external to the hollow body. The at least one valve member is movable relative to the hollow body for varying the size of an open area of the at least one air entrainment port, wherein fluid communication between the internal chamber and the space external to the hollow body via the at least one air entrainment port is permitted only via the open area of the at least one air entrainment port.

The patient may select the position of the at least one valve member to control the open area of the at least one air entrainment port. The patient may do so with a view to regulating the pressure signal detected via the gas supply tube by a pulse-flow gas source, such as a POC. In general, the magnitude of the detected pressure signal will increase as the open area of the at least one air entrainment port decreases. The patient may also do so with a view to regulating the resistance to inhalation. In general, the resistance to inhalation increases as the open area of the at least one air entrainment port decreases.

In embodiments of the nasal interface apparatus, the at least one inlet comprises a pair of inlets. In embodiments of the nasal interface apparatus, the at least one gas outlet comprises a pair of outlets. In embodiments of the nasal interface apparatus, the at least one air entrainment port may be a single air entrainment port, a pair of air entrainment ports, or more than two air entrainment ports. In embodiments of the nasal interface apparatus, the at least one valve member may be a single valve member, a pair of valve members, or more than two valve members. In embodiments of the nasal interface apparatus, the at least one inlet is oriented to direct the gas from the gas supply tube into the internal chamber in a direction towards the midline of the patient, in use when the nasal inserts are attached to the hollow body to permit fluid communication between the internal chamber and the nostrils, and received within the patient's nostrils.

In embodiments of the nasal interface apparatus, the at least one air entrainment port is disposed below the at least one outlet, in use when the nasal inserts are attached to the hollow body to permit fluid communication between the internal chamber and the nostrils, and received within the patient's nostrils, and the patient's nostrils are facing downwards.

In embodiments of the nasal interface apparatus, the at least one valve member is disposed within the internal chamber. In embodiments of the nasal interface apparatus, the at least one valve member is disposed outside of the internal chamber.

In embodiments of the nasal interface apparatus, the at least one valve member is movable by translation relative to the hollow body for varying the open area of the at least one air entrainment port.

In embodiments of the nasal interface apparatus, the nasal interface apparatus further comprises a worm gear in driving engagement with the at least one valve member for moving the at least one valve member relative to the hollow body for varying the open area of the at least one air entrainment port. The worm gear may comprise a knob for rotating the worm gear. The worm gear may define an aperture for receiving a locking pin, wherein when the locking pin is received in the aperture, the locking pin engages a part of the apparatus to limit or prevent rotation of the worm gear.

In embodiments of the nasal interface apparatus, the at least one valve member defines a tab or a groove for receiving a force applied by the patient's finger for moving the at least one valve member relative to the hollow body for varying the open area of the at least one air entrainment port.

In embodiments of the nasal interface apparatus, the valve member is movable relative to the hollow body for varying the size of the open area of the at least one air entrainment port, in response to air flow through the at least one air entrainment port, wherein the valve member is configured to move to increase the open area of the at least one air entrainment port as the flow rate of the air flow increases. The valve member may be attached to the hollow body by a hinge, so as to be movable by pivoting relative to the hollow body for varying the open area of the at least one air entrainment port.

In embodiments of the nasal interface apparatus, the at least one air entrainment port comprises a plurality of air entrainment ports, and the at least one valve member is movable relative to the hollow body for varying the size of the collective open area of the plurality of air entrainment ports by selectively occluding one or more of air entrainment ports.

In embodiments of the nasal interface apparatus, the valve member is movable relative to the hollow body for varying the size of the open area of the at least one air entrainment port in a range between about 0 mm² to about 60 mm².

In embodiments of the nasal interface apparatus, the nasal interface apparatus further comprises the pair of tubular nasal inserts attached to the manifold, for permitting fluid communication between the internal chamber and the patient's nostrils via the at least one outlet. The pair of tubular nasal inserts may comprise a pair of nasal pillows.

### BRIEF DESCRIPTION OF THE DRAWINGS

In the drawings, like elements may be assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention.

FIG. 1 shows a disassembled exploded top-front perspective view of an embodiment of a nasal interface apparatus of the present invention.

FIG. 2 shows an assembled top half-sectional view of the apparatus of FIG. 1, showing internal parts of the apparatus, along section line 1-1 of FIG. 4.

FIG. 3 shows an assembled top view of the apparatus of FIG. 1.

FIG. 4 shows an assembled front view of the apparatus of FIG. 1.

FIG. 5 shows an assembled bottom view of the apparatus of FIG. 1.

FIG. 6 shows a top view of the manifold of the apparatus of FIG. 1.

FIG. 7 shows a front view of the manifold of FIG. 6.

FIG. 8 shows a bottom view of the manifold of FIG. 6.

FIG. 9 shows a left side view of the manifold of FIG. 6.

FIG. 10 shows a top sectional view of the manifold of FIG. 6 along section line 2-2 of FIG. 7.

FIG. 11 shows a front sectional view of the manifold of FIG. 6 along section line 3-3 of FIG. 6.

FIG. 12 shows a detail view of the manifold of FIG. 6 in region 4 of FIG. 7.

FIG. 13 shows a top view of one of the valve members of the apparatus of FIG. 1.

FIG. 14 shows a front view of the valve member of FIG. 13.

FIG. 15 shows a side view of the valve member of FIG. 13.

FIG. 16 shows a side view of one of the guide members of the apparatus of FIG. 1.

FIG. 17 shows a front view of the guide member of FIG. 16.

FIG. 18 shows a top view of a worm gear of the apparatus of FIG. 1.

FIG. 19 shows a front view of a locking pin of the apparatus of FIG. 1.

FIG. 20 shows a top-rear perspective view of the apparatus of FIG. 1, with a pair of nasal pillows, and a pair of gas supply tubes attached thereto to form a system of the present invention.

FIG. 21 shows a bottom-front quarter perspective view of the system of FIG. 20 when fitted on a replica of a human face.

FIG. 22 shows a bottom view of the system of FIG. 20, with the valve members in a position corresponding to a minimum size of open area of the air entrainment ports.

FIG. 23 shows a bottom view of the system of FIG. 20, with the valve members in a position corresponding to an intermediate size of open area of the air entrainment ports.

FIG. 24 shows a bottom view of the system of FIG. 20, with the valve members in a position corresponding to a maximum size of open area of the air entrainment ports.

FIG. 25 shows a photograph of a setup for an experiment conducted on the apparatus of FIG. 1, including a replica of a human face, a pair of supply tubes, and a pair of manometers.

FIG. 26 shows a photograph of a vacuum used in conjunction with the experiment setup of FIG. 25.

FIG. 27 shows a photograph of flow meter and a valve used in conjunction with the experiment setup of FIG. 25.

FIG. 28 is a chart of signal pressure versus the open area of the air entrainment ports in an experiment conducted on the apparatus of FIG. 1, for test subject “2v1”.

FIG. 29 is a chart of signal pressure versus the open area of the air entrainment ports in an experiment conducted on the apparatus of FIG. 1, for test subject “5v0”.

FIG. 30 is a chart of signal pressure versus the open area of the air entrainment ports in an experiment conducted on the apparatus of FIG. 1, for test subject “8v0”.

FIG. 31 is a chart of pressure drop versus the open area of the air entrainment ports in an experiment conducted on the apparatus of FIG. 1, for test subject “2v1”.

FIG. 32 is a chart of pressure drop versus the open area of the air entrainment ports in an experiment conducted on the apparatus of FIG. 1, for test subject “5v0”.

FIG. 33 is a chart of pressure drop versus the open area of the air entrainment ports in an experiment conducted on the apparatus of FIG. 1, for test subject “8v0”.

FIG. 34 are tables summarizing the results of an experiment conducted on the apparatus of FIG. 1 at different open flow areas of the air entrainment ports, in comparison with a regular cannula and no cannula, for test subject “2v1”.

FIG. 35 are tables summarizing the results of an experiment conducted on the apparatus of FIG. 1 at different open flow areas of the air entrainment ports, in comparison with a regular cannula and no cannula, for test subject “5v0”.

FIG. 36 are tables summarizing the results of an experiment conducted on the apparatus of FIG. 1 at different open flow areas of the air entrainment ports, in comparison with a regular cannula and no cannula, for test subject “8v0”.

FIG. 37 is a table summarizing settings of the open flow area of air entrainment ports for the apparatus of FIG. 1, to regulate the signal pressure to a desired level at different respiratory flow rates of the patient, while keeping the pressure drop as low as possible.

FIG. 38 shows a bottom schematic view of a first alternative embodiment of a nasal interface apparatus of the present invention, with the valve member in a position corresponding to an intermediate size of open area of the air entrainment ports.

FIG. 39 shows a bottom schematic view of the apparatus of FIG. 38, with the valve member in a position corresponding to a zero size of open area of the air entrainment ports.

FIG. 40 shows a bottom schematic view of a second alternative embodiment of a nasal interface apparatus of the present invention, with the valve member in a position corresponding to a maximum size of open area of the air entrainment ports.

FIG. 41 shows a bottom schematic view of the apparatus of FIG. 40, with the valve member in a position corresponding to an intermediate size of open area of the air entrainment ports.

FIG. 42 shows a bottom schematic view of the apparatus of FIG. 40, with the valve member in a position corresponding to a zero size of open area of the air entrainment ports.

FIG. 43 shows a top front perspective view of an alternative embodiment of a manifold which may be used for an apparatus of the present invention.

FIG. 44 shows a top view of the manifold of FIG. 43.

FIG. 45 shows a bottom view of the manifold of FIG. 43.

FIG. 46 shows a rear view of the manifold of FIG. 43.

FIG. 47 shows a right side view of the manifold of FIG. 43.

FIG. 48 shows a front view of the manifold of FIG. 43.

FIG. 49 is a table summarizing the signal pressures measured for experimental Subject 2, when using a prototype apparatus of the present invention at different settings of the open flow area of the air entrainment ports, in comparison with using a standard nasal cannula.

FIG. 50 is a table summarizing the signal pressures measured for experimental Subject 9, when using a prototype apparatus of the present invention at different settings of the open flow area of the air entrainment ports, in comparison with using a standard nasal cannula.

FIG. 51 is chart of the flow rate of oxygen pulses of a POC, and the oxygen concentration waveform measured for experimental Subject 9, when using a standard nasal cannula.

FIG. 52 is chart of the flow rate of oxygen pulses of a POC, and the oxygen concentration waveform measured for experimental Subject 9, when using a prototype apparatus of the present invention.

FIG. 53 is a chart comparing the fractions of inspired oxygen measured for experimental Subject 9, when using a prototype apparatus of the present invention, at different settings of the open flow area of the air entrainment ports, in comparison with using a standard nasal cannula.

FIG. 54 is a table summarizing peak inspiratory pressure drops across a prototype apparatus of the present invention and an airway replica, for different settings of the open flow area of the air entrainment ports.

### DETAILED DESCRIPTION OF EMBODIMENTS OF THE INVENTION

The present invention relates to a nasal interface apparatus for delivering gas to a human via a gas supply tube and a pair of tubular nasal inserts.

### Definitions

Any term or expression not expressly defined herein shall have its commonly accepted definition understood by a person skilled in the art. As used herein, the following terms have the following meanings.

“Nasal insert”, as used herein, refers to a tubular member that may be received in a patient's nostril to direct a gas into the patient's nostril. Non-limiting types of nasal inserts include nasal prongs, and nasal pillows, as are known to persons skilled in the art of respiratory devices.

“Patient”, as used herein, includes a human being.

### Nasal Interface Apparatus.

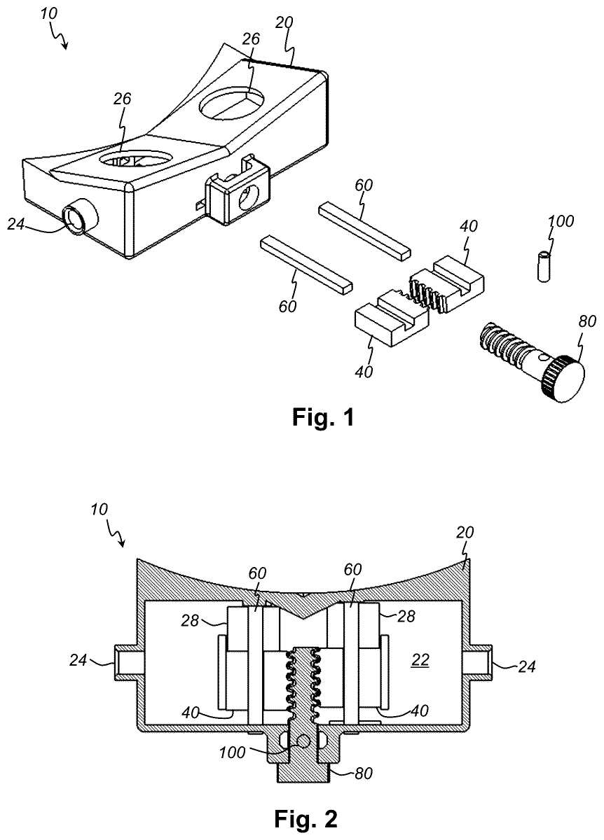

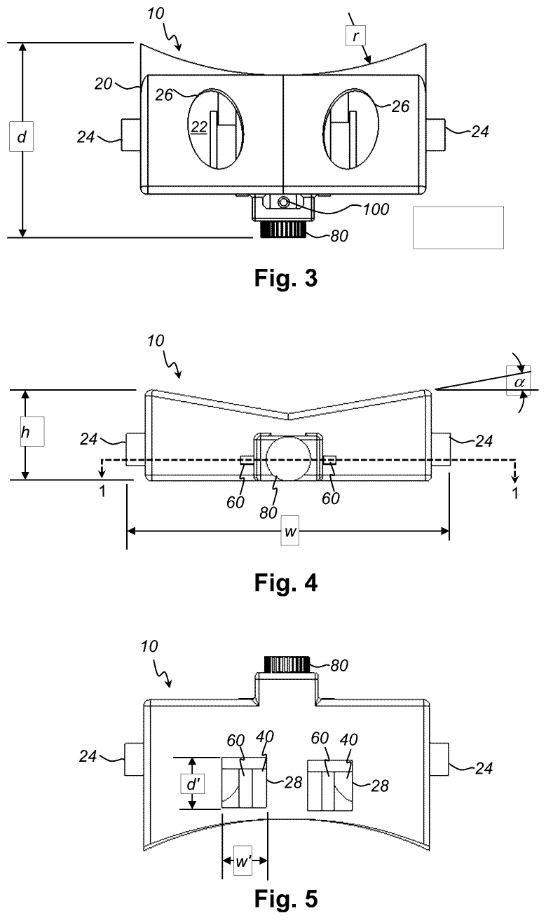

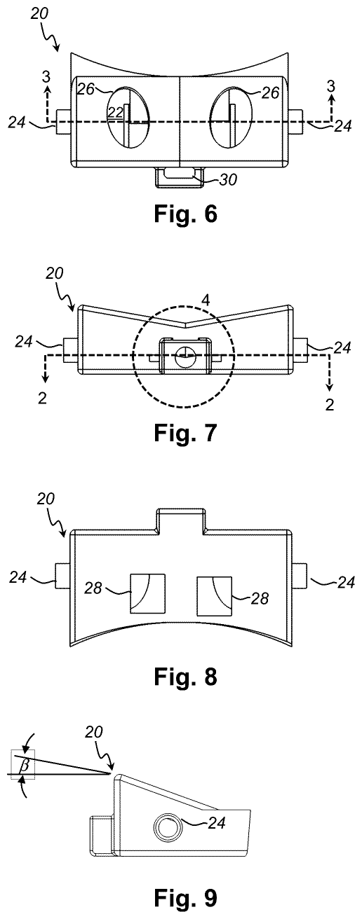

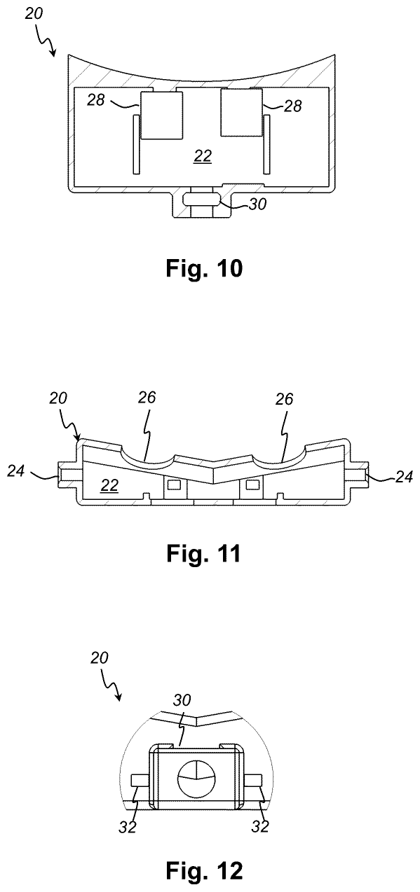

FIGS. 1 to 5 show views of an embodiment of a nasal interface apparatus (10) of the present invention. FIGS. 6 to 19 show views of constituent parts of the apparatus (10) of FIG. 1. For convenient reference, the term “longitudinal” refers to the horizontal direction aligned with the axis extending from the front to rear of the apparatus (10), while the term “transverse” refers to the horizontal direction perpendicular to the longitudinal direction. As a non-limiting example, the geometry of the embodiment of the apparatus (10) is as follows: a longitudinal depth (d) of about 30.5 mm (see FIG. 3); a height (h) of about 14.4 mm (see FIG. 4); a transverse width (w) of about 51 mm (see FIG. 4); a rear surface with a radius of curvature (r) of about 53.4 mm (see FIG. 3); an upper surface oriented at an angle (α) of about 10° relative to a transverse axis (see FIG. 4) and at an angle (β) of about 20° relative to a longitudinal axis (see FIG. 5); elliptical outlets (26) having major and minor axes with lengths of about 12.4 mm and about 8.8 mm, respectively (see FIG. 6); and rectangular air entrainment ports (28) having a longitudinal depth (d′) and transverse width (w′) of about 8 mm and about 7 mm, respectively (see FIG. 5). The dimensions of parts the apparatus (10) shown in these drawings are derivable by proportional relationship within and between the drawings. It will be understood that different embodiments of the apparatus (10) may have different geometries (shape and dimensions) so as to be adapted for use by patients of different sizes.

Referring to FIG. 1, the embodiment of the apparatus (10) includes a manifold including a hollow body (20), a pair of valve members (40), a pair of guide members (60), a worm gear (80), and a locking pin (100). In one embodiment, these parts are 3D-printed in resin material (e.g., Vero™; Stratasys Ltd., MN, USA). In other embodiments, these parts may be produced by other methods using different materials suitable for interfacing with a patient. These parts of the apparatus (10) are described in greater detail below.

### Manifold.

A purpose of the manifold is to collect the gas to be delivered to the patient's nostrils from a gas supply tube, and direct it to the patient's nostrils. Another purpose of the manifold is to allow for entrainment of room air with the gas delivered to the patient's nostrils when the patient inhales, and to allow the patient to exhale through the nostrils into the room air.

FIGS. 6 to 12 show views of the manifold of the apparatus (10) of FIG. 1. The manifold includes a hollow body (20). In the embodiment shown in the Figures, the hollow body (20) defines an internal chamber (22), a pair of inlets (24), a pair of outlets (26), and a pair of air entrainment ports (28), generally arranged in a symmetric manner about the longitudinal midline of the manifold. In other embodiments (not shown), the hollow body (22) may define only one inlet (24), one outlet (26), and one air entrainment port (28), or a greater number of them. Although certain apertures are nominally referred to as “inlets,” “outlets,” and “air entrainment ports” for convenient reference, it will be appreciated from the description of their use and operation below that such nomenclature does not limit gas flow through them to a particular direction in respect to the internal chamber (22). Each of inlets (24) as described herein may alternatively be referred to as a “first aperture”. Each of the outlets (26) as described herein may alternatively be referred to as a “second aperture.” Each of the air entrainment ports (28) as described herein may alternatively be referred to as a “third aperture.”

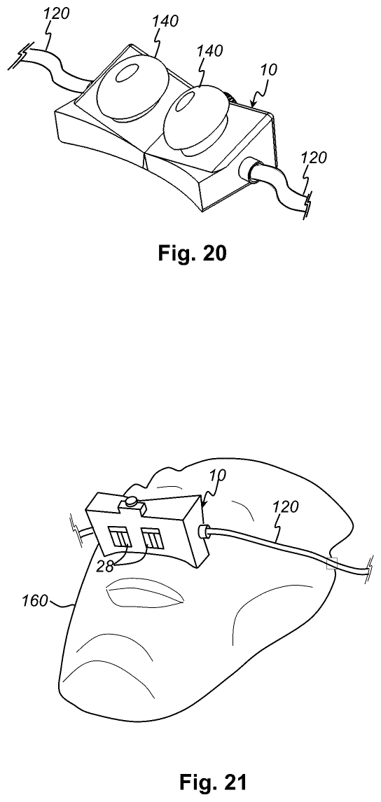

In the embodiment shown in the Figures, the hollow body (20) has a generally rectangular prismatic shape, and is sized to be worn in abutment with the portion of the patient's face between the patient upper lip and the patient's nostrils. The rear surface of the hollow body (20) is concavely arcuate to conform comfortably to that portion of the patient's face. The upper surfaces of the hollow body (20) form a shallow-angled V-shape to orient nasal inserts (140) (see FIG. 20) comfortably into the patient's nostrils.

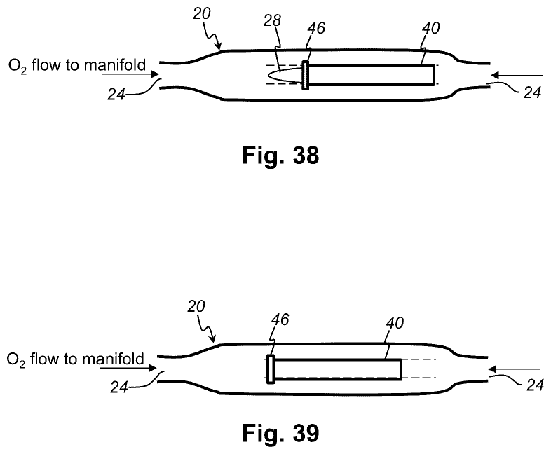

In the embodiment shown in the Figures, the hollow body (20) contains the valve members (40). In other embodiments (such as the alternative embodiments shown in FIGS. 38 to 42, as discussed below), the valve members (40) may be disposed outside of the hollow body (20).

The internal chamber (22) provides a single space through which the supplied gas and inhaled entrained air must flow before reaching the patient's nostrils.

The inlets (24) (each of which may be referred to a “first aperture” as noted above) allow for fluid communication from gas supply tubes (120) into the internal chamber (22). In the embodiment shown in the Figures, the inlets (24) are disposed on the sides of the hollow body (20) such that the outlets (26) and the air entrainment ports (28) are disposed horizontally between the inlets (24) in the transverse direction. In the embodiment shown in the Figures, the portions of the hollow body (20) that define the inlets (24) project transversely outward from the remainder of the hollow body (20) so as to provide a coupling into which plastic supply tubing can be push-fit and retained by friction fit.

The outlets (26) (each of which may be referred to as a “second aperture” as noted above) allow for fluid communication between the internal chamber (22) and the pair of nasal inserts (140) to be attached to the manifold (e.g., see FIG. 20). Thus, the outlets (26) allow the supplied gas mixed with entrained air to flow through them from the internal chamber (20) to the nasal inserts (140). The outlets (26) also allow air exhaled by the patient to flow through them from the nasal inserts (140) into the internal chamber (22). In the embodiment shown in the Figures, the outlets (26) are formed on the upper surfaces of the hollow body (20), have an elliptical shape, and are sized to receive the lower end of a nasal insert (140) in the form of a nasal pillow, and securely retain the nasal pillow by friction fit (see FIG. 20). Thus, the outlets (26) are oriented upwardly towards the patient's nostrils when the apparatus (10) is worn by the patient, when the patient's nostrils are facing downwards (e.g., when the patient is standing erect).

The air entrainment ports (28) (each of which may be referred to as a “third aperture” as noted above) allow for fluid communication between the internal chamber (22) and a space external to the hollow body (20). Thus, the air entrainment ports (28) allow room air to be drawn into the internal chamber (22) and mix with the gas supplied by gas supply tubes (120) via the inlets (24) when the patient inhales. The air entrainment ports (28) also allow air exhaled by the patient through the nasal inserts (140) and into the internal chamber (22), to exit the internal chamber (22) into the space external to the hollow body (20). In the embodiment shown in the Figures, the air entrainment ports (28) are formed in the lower surface of the hollow body (20), and disposed beneath the outlets (26). Thus, the air entrainment ports (28) are oriented downwardly when the apparatus (10) is worn by the patient, when the patient's nostrils are facing downwards (e.g., when the patient is standing erect).

### Valve Member.

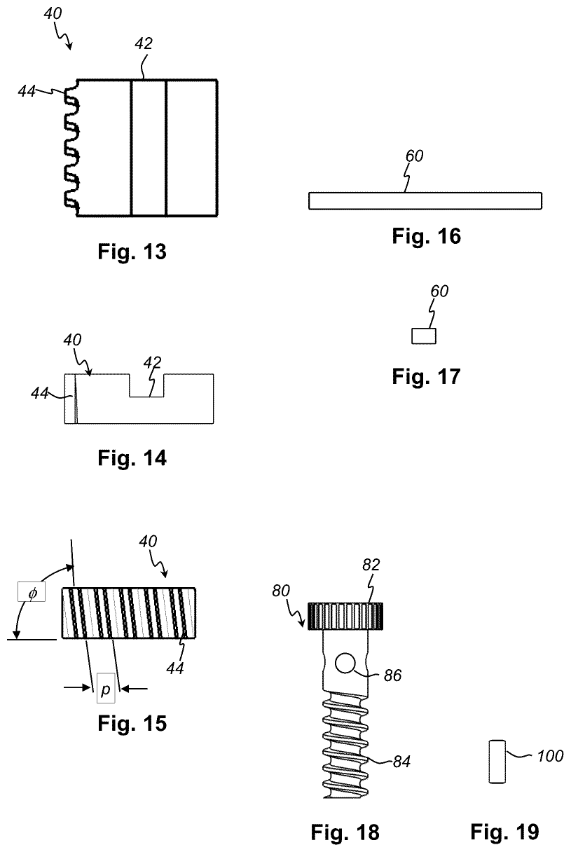

FIGS. 13 to 15 show views of one of the valve members (40) of the apparatus (10) of FIG. 1. The valve members (40) move relative to the hollow body (20) for varying the size of the open areas of the air entrainment ports (28). As used herein, “open area” refers to the portion of the air entrainment port (28) that is not occluded or otherwise obstructed by a valve member (40), so as to permit fluid communication between the internal chamber (22) and the space external to the hollow body (20) via the air entrainment ports (28). As used herein, “the closed area” refers to the portion of the area of the air entrainment port (28) through which fluid communication between the internal chamber (22) and the space external to the hollow body (20) is prevented by a valve member (40).

In one embodiment, the size of the open area may be varied from 0% to 100%, or a value in between, of the area of the air entrainment ports (28). In one embodiment, the collective open area of the air entrainment ports (28) can be varied from about 0 mm² to about 60 mm², or a range of areas in between about 0 mm² to about 60 mm².

In the embodiment shown in the Figures, each of the valve members (40) is in the form of a substantially rectangular prismatic block, and is disposed in the internal chamber (22) of the hollow body (20) above one of the air entrainment ports (28). The valve member (40) is sized so that it can translate within the internal chamber (22) in the longitudinal direction of the hollow body (20), and thereby occlude the associated air entrainment port (28) in varying degrees. In other embodiments (not shown), the valve member (40) may move relative to the hollow body (20) to vary the open area of the air entrainment ports (28) in a different direction or in a different manner, such as by rotational movement or by pivoting movement. The present invention is not limited by the movement path of the valve member (40) relative to the holly body (20).

### Guide Members.

In the embodiment of the valve member (40) shown in FIGS. 13 and 14, the upper surface of the valve member (40) defines a groove (42) extending longitudinally from the front of the valve member (40) to the rear of the valve member (40). The groove (42) is sized to receive, within close tolerances, one of the guide members (60), as shown in views in FIGS. 16 and 17. Each one of the guide members (60) is inserted into the hollow body (20) through an aperture (32) (see FIG. 12) formed on front surface of the hollow body (20), and is thereby securely retained inside the internal chamber (22), in fixed relationship to the hollow body (20). Engagement of the groove (42) with the guide member (60) limits movement of the engaged valve member (40) to translational movement in the longitudinal direction of the hollow body (20), along the guide member (60).

### Worm Gear.

In the embodiment of the valve member (40) shown in FIGS. 13 and 14, the side surface of the valve member (40) oriented toward the median of the hollow body (20) defines a series of gear teeth (44), so that the valve member (40) serves as a block gear. FIG. 18 shows a top view of the worm gear (80) of the apparatus (10) of FIG. 1. The worm gear (80) has a knob (82) at its front end, and a geared portion (84). Referring to FIG. 2, the worm gear (80) passes through an aperture defined by the hollow body (20) so that its front end knob (82) is disposed outside of the hollow body (20), and its geared portion (84) is disposed inside the hollow body (20) transversely between the valve members (40), and in driving engagement with the gear teeth (44) of the valve members (40). Accordingly, the patient may rotate the knob (82) to rotate the worm gear (80), and thereby cause longitudinal translational movement of the valve members (40), in unison, relative to the hollow body (20) to vary the open areas of the air entrainment ports (28). The gear teeth (44) and the worm gear (80) may be configured to allow the patient to make fine adjustments in the position of the valve members (40). As a non-limiting example, the pitch (p) of the gear teeth (40) may be about 1.5 mm, and the gear teeth may be oriented at an angle (ϕ) of about 83.2° (see FIG. 15).

### Locking Pin.

In the embodiment of the worm gear (80) shown in FIG. 18, the front end of the worm gear (80) defines a worm gear aperture (86) that removably receives the locking pin (100) (FIG. 19). In the embodiment shown in FIG. 6, the front end of the manifold defines a manifold pocket (30) that is sized to receive the lower end of the locking pin (100). When the locking pin (100) is removed from the worm gear aperture (86), the worm gear (80) is free to rotate, and thereby drive movement of the valve members (40) as described above. Conversely, when the locking pin (100) is received in the worm gear aperture (86), the locking pin (100) interferes with the walls defining the manifold pocket (30) so as to prevent or limit inadvertent rotation of the worm gear (80), and thus prevent or limit movement of the valve members (40). In this manner, the position of the valve members (40) can be selectively fixed by the patient.

### Use and Operation of Apparatus.

The use and operation of the embodiment of the apparatus (10) of FIG. 1 is now described. As shown in FIG. 20, the apparatus (10) is prepared for use by attaching a pair of gas supply lines (120) to portion of the hollow body (20) defining the inlets (24), so that the inlets (24) permit fluid communication from the gas supply lines (120) to the internal chamber (22) of the hollow body (20). The apparatus (10) is further prepared for use by attaching a pair of nasal inserts (140) to the hollow body (20), so that the nasal inserts (140) are in fluid communication with the internal chamber (22) of the hollow body (20) via the outlets (26).

In the embodiment shown in FIG. 20, the nasal inserts (140) are in the form of nasal pillows, which are adapted to engage with the inner wall of the patient's nostrils when inserted therein. In other exemplary uses, the nasal inserts (140) may be in the form of nasal prongs. However, the ratio of the patient's nostril area to the nasal insert area may affect the efficacy of varying the open area of the air entrainment ports (28) for the purposes described below. It may be preferable for this ratio to be lower, and as close to 1:1 possible. As such, the use of nasal pillows that engage the inner wall of the patient's nostrils, may be preferable to the use of nasal prongs that typically do not engage the inner wall of the patient's nostrils.

As shown in FIG. 21 using a replica (160) of a human face, the apparatus (10) is worn such that the concave rear surface of the hollow body (20) engages the portion of the patient's face between the upper lip and nostrils. The nasal inserts (140) are inserted into the patient's nostrils. When worn in this manner, the inlets (24) are oriented to direct the gas from the gas supply tubes (120) into the internal chamber (22) in a substantially transverse direction towards the midline of the patient, while the air entrainment ports (28) are disposed below the outlets (26), when the patient's nostrils are facing downwards (e.g., when the patient is standing erect).

The gas supply lines (120) are connected to a gas source (not shown). In a non-limiting exemplary use, the gas source may be a portable pulse-flow portable oxygen concentrator (POC). In other exemplary uses, the gas source may or may not be portable, may supply oxygen or another gas, and may supply the gas in pulse-flow or continuous flow. The use of the present invention is not limited by the nature of the gas source. In use, the gas supply lines (120) deliver oxygen from the pulse-flow POC to the internal chamber (22) of the hollow body (20) via the inlets (24).

When the patient inhales, the suction through the patient's nostrils draws room air from the space external to the hollow body (20) into the internal chamber (22) via the air entrainment ports (28). The gas source (not shown) supplies gas through the supply tubes (120) into the internal chamber (22) via the inlets (24). In the internal chamber (22), the supplied gas and the entrained air mix together. The mixture is drawn from the internal chamber (22) into the patient's nostrils via the outlets (26) and the attached nasal inserts (140). When the patient exhales, the exhaled air flows from the patient's nostrils into the internal chamber (22) via the nasal inserts (140) and the outlets (26), and out of the internal chamber (22) via the air entrainment ports (28).

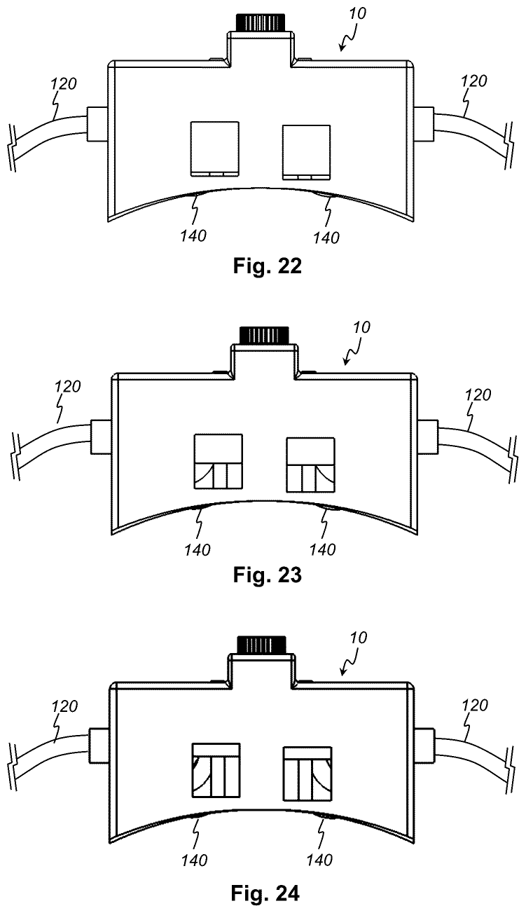

As shown in FIGS. 22 to 24, the patient may rotate the worm gear (80) to move the valve members (40) to the rear-most position such that the open area of the air entrainment ports (28) are at a minimum size (e.g., at about 0% of the area of the air entrainment ports (28)) (FIG. 22), to an intermediate position such that the open area of the air entrainment ports (28) are at an intermediate size (e.g., between about 0% and about 100% of the area of the air entrainment ports (28) (FIG. 23), or to a front-most position such that the open areas of the air entrainment ports (28) are at a maximum size (e.g., at or about 100% of the area of the air entrainment ports (28)) (FIG. 24).

When the apparatus (10) is used with a pulse-flow POC, one purpose of varying the open area of the air entrainment ports (28) is to control the signal pressure detected by the pulse-flow POC at its outlet to the gas supply tubes (120) upon patient inhalation. In general, as the size of the open area of the air entrainment ports (28) decreases, the resistance to flow through them increases. Accordingly, as the size of the open area of the air entrainment ports (28) decreases, it can be expected that a lower negative pressure (relative to the room ambient pressure) will develop within the internal chamber (22) in order to draw room air through the air entertainment ports (28), and the signal pressure detected in the gas supply tubes (120) by the pulse-flow POC will tend to increase in absolute value.

By exploiting this principle of operation, the apparatus (10) may be used to regulate the signal pressure detected by the pulse-flow POC at a level necessary for the pulse-flow POC to detect patient inhalation, despite changes in respiratory flow rate (e.g., higher respiratory flow rate when the patient is active during the day time, versus low respiratory flow rate when the patient is sleeping at night time). The size of the open area or the air entrainment ports (28) may be adjusted until the patient or his or her caregiver receives visual and/or auditory confirmation that the pulse-flow POC is triggering and sending pulses of oxygen, while having regard to the patient's perception of any imposed resistance to breathing.

### Experimental Example of Use of Apparatus

An experiment was conducted on the apparatus (10) of FIG. 1 to study the effect of varying the open area of the air entrainment ports (28) on the signal pressure detectable through gas supply lines (120), and the pressure drop. The pressure drop refers to the change in inhalation pressure in the airway as measured at the nostril. The pressure drop is an important parameter because a higher pressure drop is indicative of the apparatus (10) imposing a higher resistance to respiratory flow. If the resistance is too great, it may become uncomfortable or difficult for the patient to breath. Of course, the size of the open area of the air entrainment ports (28) should be sufficiently large that any imposed resistance to inspiratory or expiratory flow is acceptable to the patient.

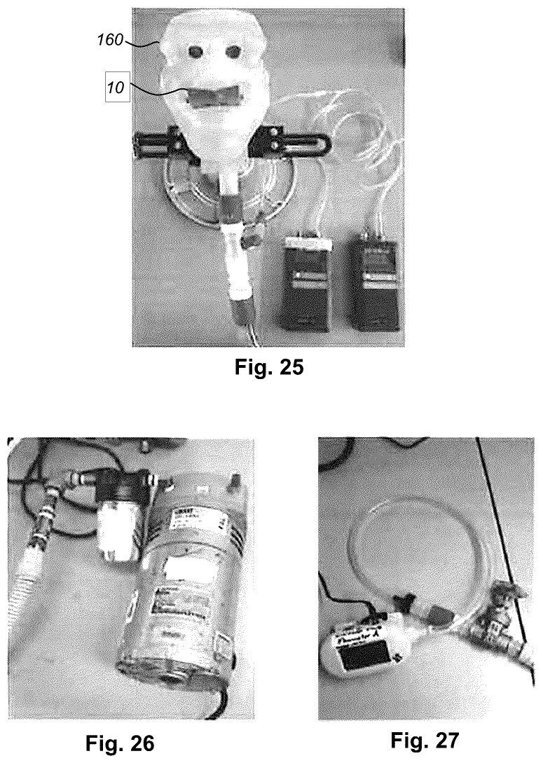

FIGS. 25 to 27 show the set up used to conduct the experiment. The apparatus (10) was used with nasal pillows and fitted to three different replicas (160) of the human face and upper airways comprising the nasal cavity, nasopharynx, larynx, and entrance to the trachea (FIG. 25), identified as subjects “2v1”, “5v0”, and “8v0”. The nasal pillows fit well within the replica (160) nostrils. A vacuum (FIG. 26) was connected to each replica (160) using hoses to simulate patient respiratory inhalation. A flow meter and valve (FIG. 27) was used to monitor and control the flow rate produced by the vacuum. Two manometers (Omega HHP-103™; Omega Engineering, Inc., CT, USA) (as shown in FIG. 25) were used. The first manometer was connected to the gas supply tubes to measure the signal pressure. The second manometer was placed in line with the vacuum hose, just upstream of the connection to the replica (160), to measure the pressure drop with the apparatus (10) in place.

With this setup in place, the experiment was conducted on the three face replica subjects, denoted “2v1”, “5v0”, and “8v0”, at different simulated respiratory flow rates in combination with different sizes of the open areas of the air entrainment ports (28). The experiment was also conducted on the three subjects without any nasal insert, and when fitted with a conventional nasal cannula (Hudson RCI™ over-the-ear nasal cannula; Teleflex Medical Incorporated, NC, USA).

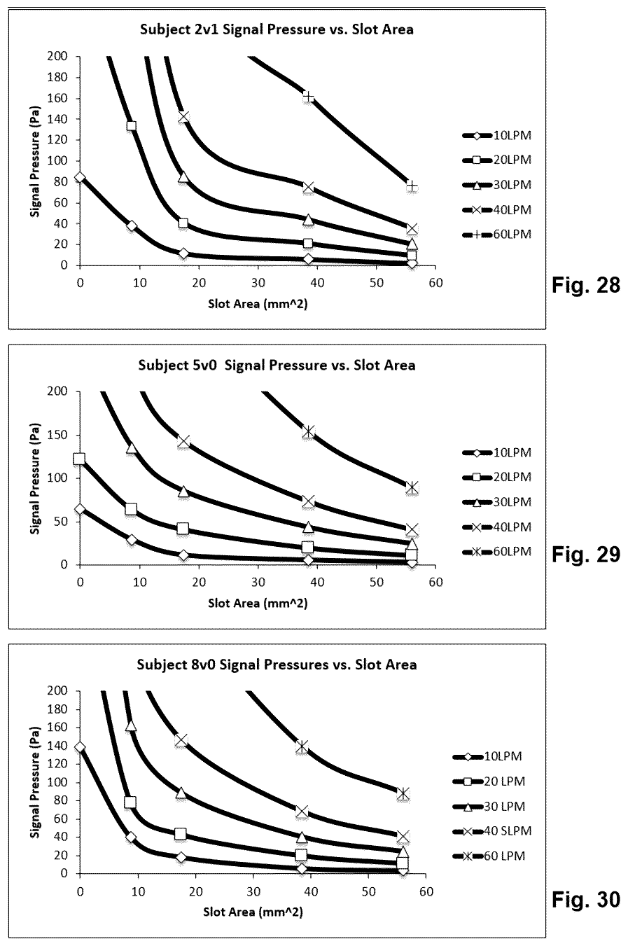

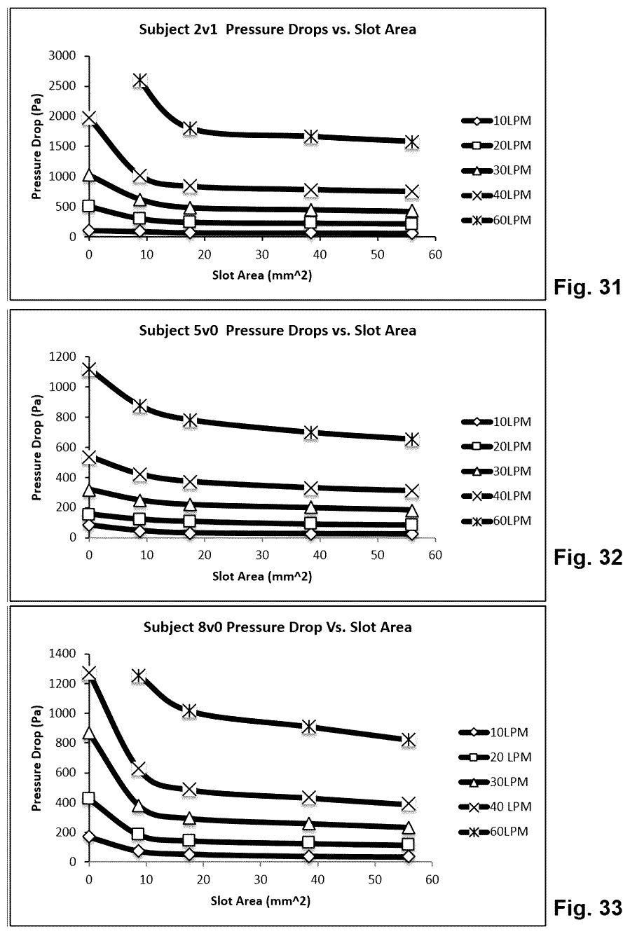

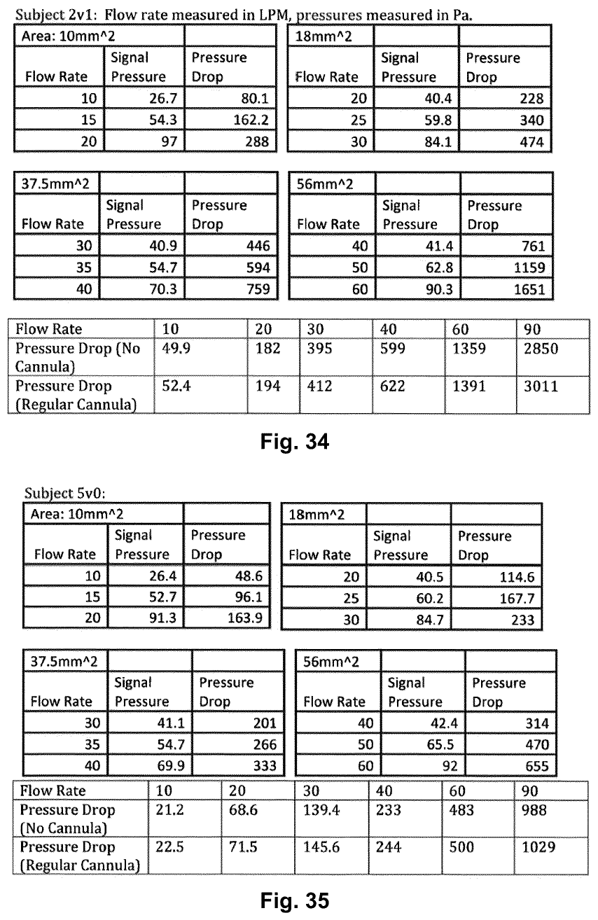

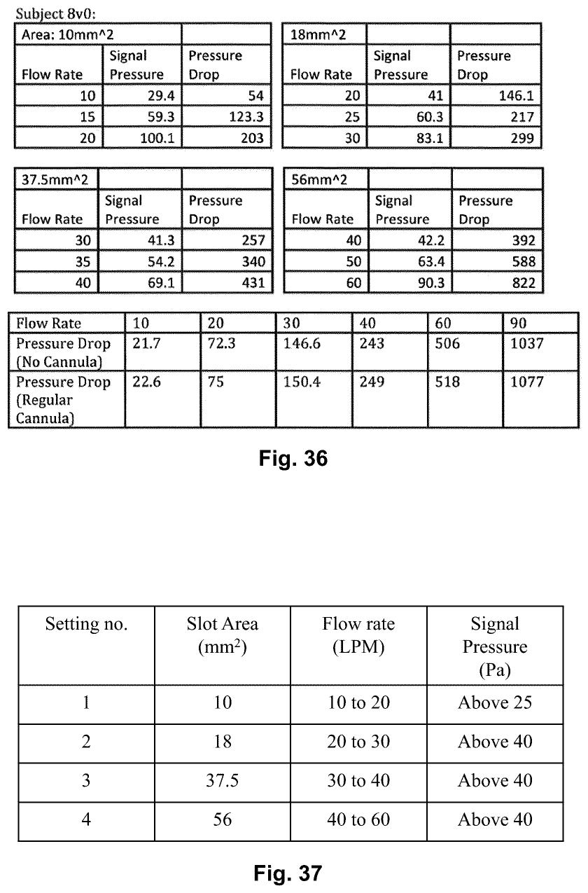

FIGS. 28 to 30 show charts of the signal pressure versus the open area of the air entrainment ports (28) (denoted “slot area” in the charts), at flow rates ranging from 10 L per minute to 60 L per minute (LPM), for subjects “2v1”, “5v0”, and “8v0”, respectively. FIGS. 31 to 33 show charts of the pressure drop versus the open area of the air entrainment ports (28) (denoted “slot area” in the charts), at flow rates ranging from 10 L per minute to 60 L per minute, for subjects “2v1”, “5v0”, and “8v0”, respectively. FIGS. 34 to 37 show tables summarizing the pressure signal data and pressure drop data shown in FIGS. 28 to 33 (as the case may be), and also pressure drop data for the subjects when tested without any nasal insert, and with the conventional nasal cannula. In these tables, the areas refer to the open flow area of one of the air entrainment ports (28); the flow rates are expressed in liters per minute (LPM); and the signal pressure and pressure drop are expressed in pascals (Pa). Although FIGS. 28 to 30, and 34 to 37 indicate the signal pressure as a positive value, it will be understood that the signal pressure is actually a negative pressure relative to ambient pressure in the room air. For example, a signal pressure of 40 Pa shown in the Figures means that the pressure detected in the gas supply tubes is 40 Pa below the ambient pressure.

Referring to FIGS. 28 to 37, the following observations and deductions may be made for all three subjects. First, for all flow rates, the signal pressure decreases as the open area of the air entrainment ports (28) increases. Second, for all flow rates, the pressure drop decreases as the open area of the air entrainment ports (28) increases. Third, for a given signal pressure, the open area of the air entrainment ports (28) and the respiratory flow rate may be approximately linearly correlated. For example, in order to maintain a signal pressure of at least 40 Pa, while minimizing imposed resistance to inspiratory/expiratory flow, the open area of the air entrainment ports (28) (in mm²) (A) can be approximately related to the respiratory flow rate (in LPM) (Q) according to the following relationship:

$$A = 1.6 \times Q - 10.5 .$$

Fourth, this embodiment of the apparatus (10) may be used to regulate the signal pressure above desired levels at different respiratory flow rates of the patient, while keeping the pressure drop as low as possible, by selective adjustment of the open areas of the air entrainment ports (28) in accordance with certain settings as shown in FIG. 37. In FIG. 37, the “slot area” refers to the open area of each of the air entrainment ports (28).

### First Alternative Embodiment of Apparatus

FIGS. 38 and 39 show a schematic illustration of a first alternative embodiment of a nasal interface apparatus (10) of the present invention. In these Figures, parts analogous to parts of the embodiment of the apparatus (10) of FIG. 1 are assigned like reference numerals. It will be appreciated that the embodiment shown in FIGS. 38 and 39 also has outlets (26), but they are not visible in the views shown. The embodiment shown in FIGS. 38 and 39 differs from the embodiment of FIG. 1 in that the valve member (40) is in the form of a single thin, elongate cover plate that slides transversely in relation to a single air entrainment port (28) having an elongate elliptical shape. The valve member (40) may be either inside or outside of the internal chamber (22). The valve member (40) has a tab or a groove (46) for the patient's finger to apply a force to slide the valve member (40) in relation to the air entrainment port (28) to vary its open area from an intermediate size (FIG. 38) to a zero size (FIG. 39), or vice versa.

### Second Alternative Embodiment of Apparatus

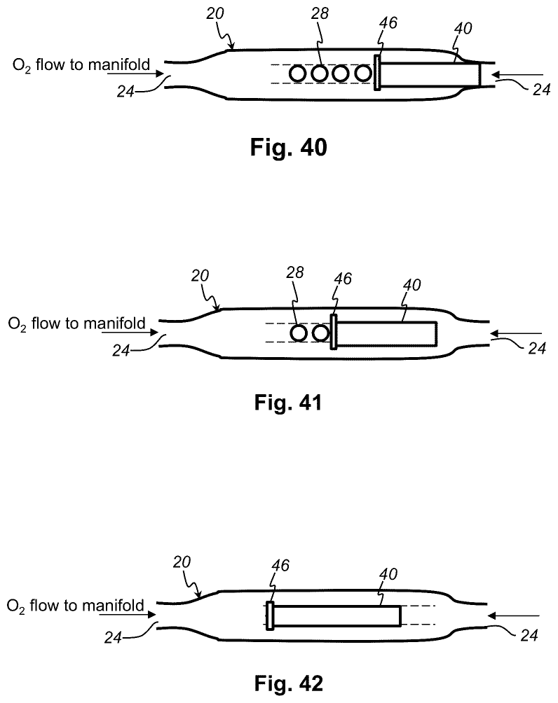

FIGS. 40 to 42 show a schematic illustration of a second alternative embodiment of a nasal interface apparatus (10) of the present invention. Parts analogous to parts of the embodiment of the apparatus (10) of FIG. 38 are assigned like reference numerals. It will be appreciated that the embodiment shown in FIGS. 38 and 39 also has outlets (26), but they are not visible in the views shown. The embodiment shown in FIGS. 40 to 42 differs from that shown in FIGS. 38 to 39, in that the valve member (40) slides in relation to a four air entrainment ports (28) to vary their collective open area from a maximum size (FIG. 40) to an intermediate size (FIG. 41) to a zero size (FIG. 42). The use of a single valve member (40) movable in relation to a plurality of air entrainment ports (28) provides the patient with a visible cue of the size of the collective open area of the air entrainment ports (28), by simply counting the number of air entrainment ports (28) that are not covered by the valve member (40), as the air entrainment ports (28) are visible from outside of the apparatus (10). For example, FIG. 40 may be considered as showing “Setting 4” because all four air entrainment ports are open, FIG. 41 may be considered as showing “Setting 2” because only two air entrainment ports are open, and FIG. 3 may be considered as showing a “Closed Setting” because all four air entrainment ports are closed.

### Third Alternative Embodiment of the Apparatus

In the embodiments of the apparatus (10) described and shown above, the patient or his or her caregiver manually manipulates the valve member (40) (e.g., by rotation of the worm gear (80) in the embodiment of FIG. 1, or by direct application of finger pressure to the valve member (40) in the embodiments of FIGS. 38 to 42) to vary the open area of the air entrainment port(s) (28). In another embodiment (not shown), the valve member (40) may move automatically (i.e., without manual intervention) in response to the respiratory flow rate through the air entrainment port(s) (28). For example, the valve member (40) may comprise a flap that is attached to the hollow body (20) by a hinge, so as to move by pivoting relative to the hollow body (20). The valve member (40) is increasingly deflected as the flow rate through the air entrainment port(s) (28) increases, so as to increase the open area of the air entrainment port(s) as the flow rate through the air entrainment port(s) (28) increases.

### Fourth Alternative Embodiment of the Apparatus

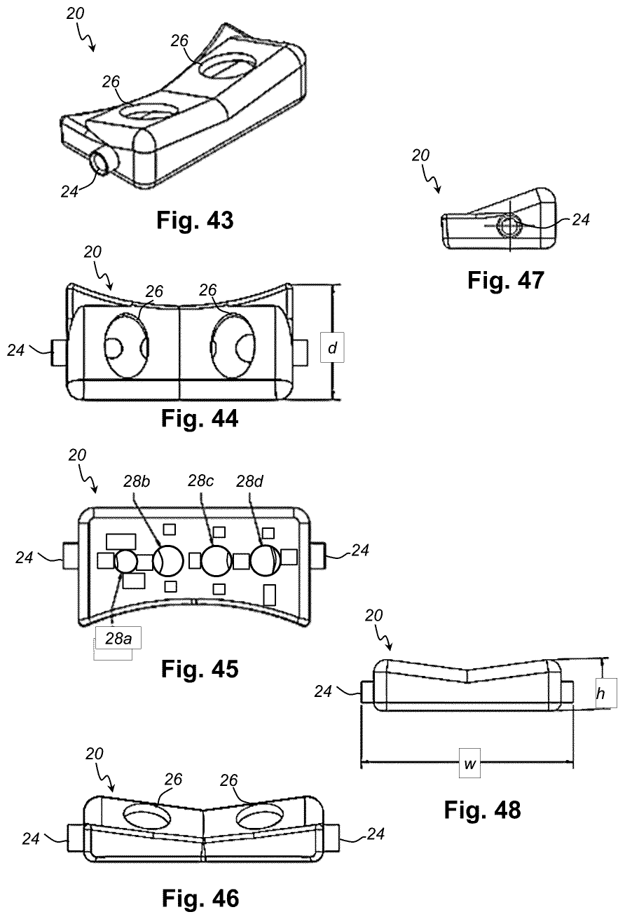

FIGS. 43 to 48 show views of an alternative embodiment of a manifold which may be used in an apparatus (10) of the present invention. In these Figures, parts analogous to parts of the embodiment of the manifold shown in FIGS. 6 to 12 are assigned like reference numerals. The use and operation of this embodiment of the manifold is the same as described above. The embodiment shown in FIGS. 43 to 48 differ in at least the following respects. The geometry of the embodiment of the apparatus (10) is as follows: a longitudinal depth (d) of about 23 mm (see FIG. 44); a height (h) of about 12 mm (see FIG. 48); a transverse width (w) of about 51 mm (see FIG. 48). The manifold defines four transversely spaced apart air entrainment ports (28a, 28b, 28c, and 28d). Circular air entrainment port (28a) has a diameter of about 4 mm, and circular air entrainment ports (28b, 28c, 28d) have a diameter of about 5.5 mm (see FIG. 48). The dimensions of other features of the manifold shown in these drawings are derivable by proportional relationship within and between the drawings.

### Additional Experimental Example of Use of Apparatus

The embodiment of the manifold shown in FIGS. 43 to 48 was used to produce a prototype apparatus (10) of the present invention. This prototype apparatus was fitted with nasal inserts (140) in the form of nasal pillows, and with gas supply tubes (120) for supplying oxygen to the prototype apparatus in a manner analogous to that described above. Experiments were conducted on the prototype apparatus to determine the following information.

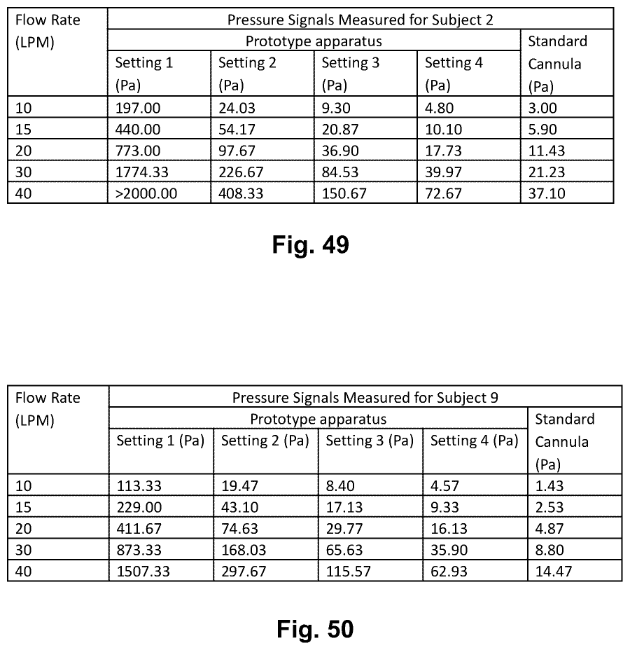

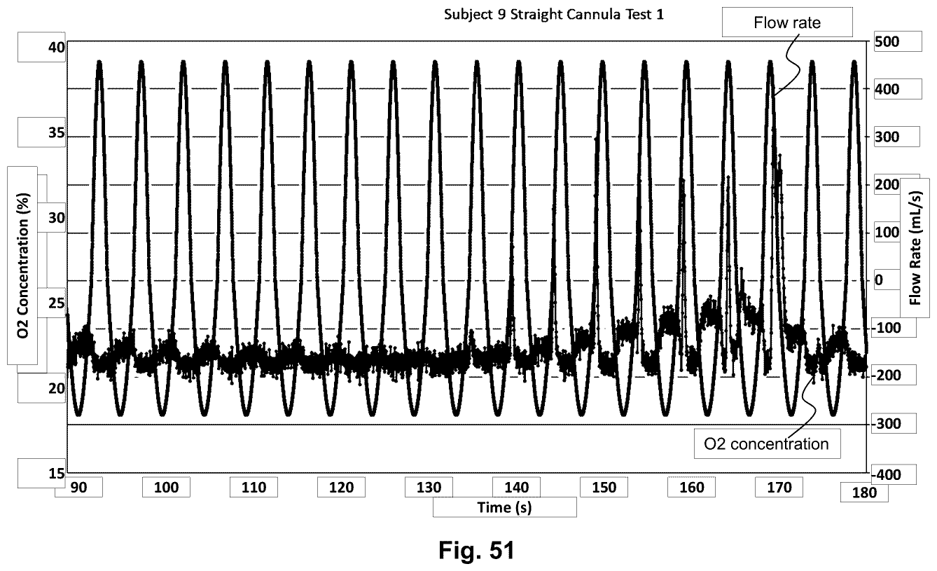

First, signal pressures were monitored on the oxygen supply tubes (120) over different inhaled flow rates. Realistic adult nasal airway replicas described previously in the study of continuous and pulsed oxygen delivery (see: Chen et al., Comparison of pulsed versus continuous oxygen delivery using realistic adult nasal airway replicas. Int J Chron Obstruct Pulmon Dis. 2017; 12:2559) were used for testing the signal pressures generated when breathing through the prototype apparatus versus a standard nasal cannula (Hudson RCI Model 1103™; Teleflex Medical, Wayne, PA, USA). The “Subject 2” replica was chosen to test as a control because this replica previously proved to generate high signal pressures, and had no issues triggering POCs when used with the standard cannula. On the other hand, the “Subject 9” replica showed low signal pressures, leading to triggering issues when used with the standard cannula. A constant flow of air at 10, 15, 20, 30 and 40 L per minute (LPM) was drawn through the airway replicas, simulating inhalation. At each flow rate the signal pressure detected by a manometer positioned at the end of the oxygen tubing supplying the prototype apparatus or standard cannula was recorded. The tables in FIGS. 49 and 50 summarize the measured signal pressures for the Subject 2 replica and the Subject 9 replica, respectively. Settings 1, 2, 3 and 4 refer to the number of open air entrainment ports (28a, 28b, 28c, 28d) on the prototype apparatus, where for setting 1 only the 4 mm diameter air entrainment ports (28a) was open for air entrainment.

Measured signal pressures can be compared with typical POC trigger pressures of about 15 to 25 Pa. While using the standard cannula, the Subject 2 replica demonstrated much higher signal pressures at all flow rates when compared to the Subject 9 replica, and exceeded typical POC trigger pressures for flow rates of 30 and 40 LPM. For the standard cannula used with the Subject 9 replica, signal pressures were below typical trigger pressures for the full range of flow rates studied. However, when using the prototype apparatus, Subject 9's signal pressures increased to values more comparable with Subject 2's. For Setting 2, signal pressures met or exceeded typical trigger pressures for both replicas at all flow rates tested. Setting 1 was not used in the following tests as the resulting signal pressures were much higher than needed to trigger typical POCs for the flow rate range studied.

Second, oxygen concentration waveforms and fractions of inspired oxygen (FiO2) were monitored for the prototype apparatus versus standard nasal cannula used with a commercial portable oxygen concentrator (POC). Since the prototype apparatus greatly increased Subject 9's signal pressures as compared with the standard cannula, it was expected that the subject 9 replica would be able to trigger a POC during breathing conditions where the standard cannula failed. Oxygen concentration waveforms were collected using methods as shown in Chen et al., 2017, supra, to test this expectation. Tests were conducted using a SimplyGo Mini™ POC (Philips Respironics; Markham, Ontario, Canada), on a pulse setting of 2. The breathing patterns used during these tests represent parameters typical of a COPD patient during sleep. As expected, the Subject 9 replica triggered a burst of oxygen from the POC while using the prototype apparatus under circumstances where the standard cannula failed.

FIG. 51 shows a waveform where the Subject 9 replica failed to trigger the POC using a standard cannula. The Subject 9 replica failed to trigger the POC using a standard cannula in 3 of 3 repeated experiments. When a patient fails to trigger the POC, the POC defaults to a timed-pulse setting, which is not in sequence with the patient's breathing. That is, the timed pulse does not always line up with the patient's inhalation. From the 90 second mark to about the 135 second mark on FIG. 51, the pulse of oxygen lines up with the exhalation phase of the simulated breath, resulting in a low oxygen concentration (%) at the trachea. At about the 140 second mark, the timed pulse starts to align better with inhalation, but this only lasts for about 7 breaths and then the pulse occurs during exhalation again.

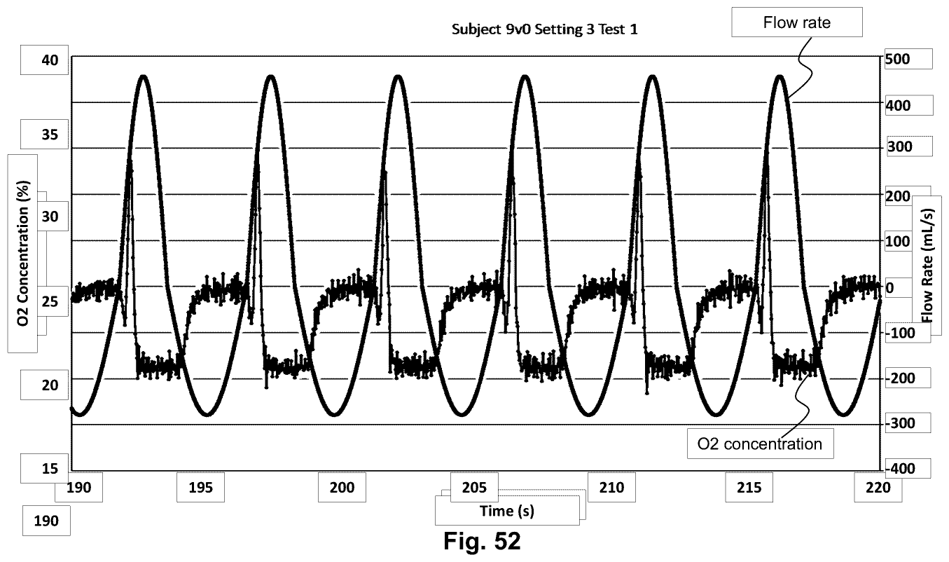

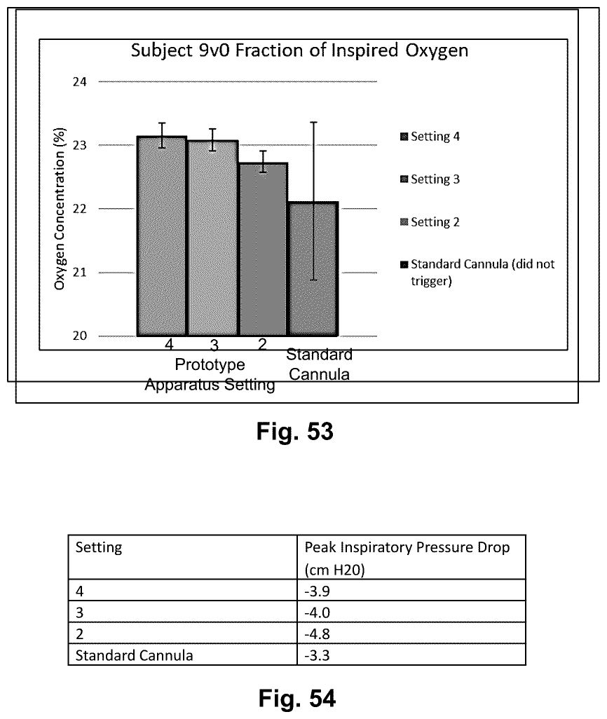

In contrast, under identical simulated breathing conditions, the Subject 9 replica triggered the POC successfully using the prototype apparatus for settings 2, 3 and 4, as described above; setting 1 was not tested. FIG. 52 shows the waveform collected while using the prototype apparatus with setting 3. The Subject 9 replica successfully triggered the POC while using the prototype apparatus for all breaths at all settings during every test conducted. As a result of successful triggering, Subject 9 showed higher and more consistent average inhaled FiO2 values when using the prototype apparatus, as shown in FIG. 53. The FiO2 values shown in FIG. 53 were averaged across a minimum of 15 inhalations for both the prototype interface at each setting and the standard cannula. The error bars are equal to the standard deviation in FiO2 over 15 inhalations.

Third, the pressure drop induced by the prototype apparatus was observed. Pressure drop refers to the additional resistance to inhalation flow. While collecting the oxygen concentration waveforms, the combined total pressure drop across the prototype apparatus and the airway replica were also recorded. As summarized by the table shown in FIG. 54, the peak negative pressures recorded while using the prototype apparatus in settings 4, 3 and 2 were −3.9 cm H₂O, −4.0 cm H₂O, and −4.8 cm H₂O, respectively. The peak negative pressure drop while using the standard nasal cannula was −3.3 cm H₂O. Settings 3 and 4 caused an additional pressure drop (above that measured for a standard nasal cannula) of less than 0.8 cm H₂O in both cases. This provides evidence that increasing the open area available for air entrainment reduces inspiratory pressure drop and hence the resistance to breathing imposed by the prototype apparatus.

### DISCLOSED EMBODIMENTS

Embodiment A of the apparatus disclosed herein includes: a manifold comprising hollow body defining; an internal chamber; at least one inlet for fluid communication from the gas supply tube into the internal chamber; at least one outlet for fluid communication between the internal chamber and the pair of nasal inserts; and at least one air entrainment port for fluid communication between the internal chamber and a space external to the hollow body; and at least one valve member movable relative to the hollow body for varying the size of an open area of the at least one air entrainment port, wherein fluid communication between the internal chamber and the space external to the hollow body via the at least one air entrainment port is permitted only via the open area of the at least one air entrainment port.

Embodiment A described above may have one or more of the following additional elements in any combination.

Element 1: a pair of inlets.

Element 2: a pair of outlets.

Element 3: one air entrainment port, or a plurality of air entrainment ports equal in number to two, or more than two.

Element 4: the at least one inlet being oriented to direct the gas from the gas supply tube into the internal chamber in a direction towards the midline of the patient, in use when the nasal inserts are attached to the hollow body to permit fluid communication between the internal chamber and the nostrils, and received within the patient's nostrils.

Element 5: the at least one air entrainment port being disposed below the at least one outlet, in use when the nasal inserts are attached to the hollow body to permit fluid communication between the internal chamber and the nostrils, and received within the patient's nostrils, when the patient's nostrils face downwards.

Element 6: the at least one valve member being disposed within the internal chamber, or being disposed outside of the internal chamber.

Element 7: the at least one valve member being movable by translation relative to the hollow body for varying the open area of the at least one air entrainment port.

Element 8: a worm gear in driving engagement with the at least one valve member for moving the at least one valve member relative to the hollow body for varying the open area of the at least one air entrainment port.

Element 9: the at least one valve member defining a tab or a groove for receiving a force applied by the patient's finger for moving the at least one valve member relative to the hollow body for varying the open area of the at least one air entrainment port

Element 10: the at least one air entrainment port comprising a plurality of air entrainment ports, and the at least one valve member being movable relative to the hollow body for varying the size of the collective open area of the plurality of air entrainment ports by selectively occluding one or more of air entrainment ports.

Element 11: the valve member being movable relative to the hollow body for varying the size of the open area of the at least one air entrainment port in a range between about 0 mm² to about 60 mm².

Element 12: the pair of tubular nasal inserts attached to the manifold, for permitting fluid communication between the internal chamber and the patient's nostrils via the at least one outlet.

Element 13: the pair of tubular nasal inserts comprising a pair of nasal pillows.

### INTERPRETATION

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

1. A system for delivering gas to a patient via the patient's nostrils, comprising:
(a) a pulse flow gas source and a gas supply line in fluid communication with a nasal interface assembly, the pulse flow gas source including a pressure sensor and configured to release a pulse of gas only when a signal pressure detected by the pressure sensor is below a trigger pressure level;
(b) wherein the nasal interface assembly comprises a pair of nasal inserts and a hollow body manifold defining:
(i) an internal chamber;
(ii) at least one inlet for fluid communication from the gas supply line into the internal chamber;
(iii) at least one outlet for fluid communication between the internal chamber and the pair of nasal inserts; and
(iv) at least one air entrainment port for fluid communication between the internal chamber and a space external to the hollow body; and
(v) at least one valve member movable relative to the hollow body for varying the size of an open area of the at least one air entrainment port, wherein fluid communication between the internal chamber and the space external to the hollow body via the at least one air entrainment port is permitted only via the open area of the at least one air entrainment port;
(c) wherein the at least one valve member is positioned to create a signal pressure below the trigger pressure upon inhalation by the patient, and above the trigger pressure upon exhalation.

2. The system of claim 1, wherein the at least one inlet comprises a pair of inlets, and the at least one gas outlet comprises a pair of outlets.

3. The system of claim 1, wherein the at least one inlet is oriented to direct the gas from the gas supply tube into the internal chamber in a direction towards the midline of the patient, in use when the nasal inserts are attached to the hollow body to permit fluid communication between the internal chamber and the nostrils, and received within the patient's nostrils.

4. The system of claim 1, wherein the at least one air entrainment port is disposed below the at least one outlet, in use when the nasal inserts are attached to the hollow body to permit fluid communication between the internal chamber and the nostrils, and received within the patient's nostrils, and the patient's nostrils are facing downwards.

5. The system of claim 1, wherein the at least one valve member is disposed within the internal chamber.

6. The system of claim 1, wherein the at least one valve member is disposed outside of the internal chamber.

7. The system of claim 1, wherein the at least one valve member is movable by translation relative to the hollow body for varying the open area of the at least one air entrainment port.

8. The system of claim 1, further comprising a worm gear in driving engagement with the at least one valve member for moving the at least one valve member relative to the hollow body for varying the open area of the at least one air entrainment port.

9. The system of claim 8, wherein the worm gear comprises a knob for rotating the worm gear.

10. The system of claim 8, wherein the worm gear defines an aperture for receiving a locking pin, wherein when the locking pin is received in the aperture, the locking pin engages a part of the apparatus to limit or prevent rotation of the worm gear.

11. The system of claim 1, wherein the at least one valve member defines a tab or a groove for receiving a force applied by the patient's finger for moving the at least one valve member relative to the hollow body for varying the open area of the at least one air entrainment port.

12. The system of claim 1, wherein the at least one air entrainment port comprises a plurality of air entrainment ports, and the at least one valve member is movable relative to the hollow body for varying the size of the collective open area of the plurality of air entrainment ports by selectively occluding one or more of air entrainment ports.

13. The system of claim 1, wherein the valve member is movable relative to the hollow body for varying the size of the open area of the at least one air entrainment port in a range between about 0 mm² to about 60 mm².

14. The system of claim 1, further comprising the pair of tubular nasal inserts attached to the manifold, for permitting fluid communication between the internal chamber and the patient's nostrils via the at least one outlet.

15. The system of claim 14 wherein the pair of tubular nasal inserts comprises a pair of nasal pillows.

16. A method of delivering a gas to a patient with a pulse flow gas source in fluid communication with a nasal interface assembly, the pulse flow gas source having a trigger pressure to release a pulse of gas and including a pressure sensor for monitoring a signal pressure in the nasal interface assembly, the method comprising the steps of:
(a) inserting the nasal interface assembly into or around the patient's nostrils;
(b) configuring the nasal interface assembly to produce a signal pressure which is lower than the trigger pressure upon inhalation by the patient; and
(c) releasing a pulse of gas to the nasal interface assembly only when signal pressure is lower than the trigger pressure.

17. The method of claim 16, wherein the nasal interface assembly signal pressure is varied by adjusting the size of an entrainment port permitting air to enter the nasal interface assembly and mix with the delivered gas.
